(19) United States
(12) Patent Application Publication
(10) Pub. No.: US 2008/0139989 A1
(43) Pub. Date: Jun. 12, 2008

(54) Hemodialysis needle device having multiple needles mounted on a needle hub

(76) Inventor: Stanley Kim, Upland, CA (US)

Correspondence Address: SHELDON MAK ROSE & ANDERSON PC, 100 Corson Street, Third Floor, PASADENA, CA, 91103-3842, US

(21) Appl. No.: 11/637,253

(22) Filed: Dec. 11, 2006

(51) Int. Cl. A61M 1/14

(52) U.S. Cl. 604/6.16

(57) ABSTRACT

A hemodialysis needle or cannula device (FIG. 1) having a plurality of a needle mounted on a hub.

Multiple thinner needles instead of a single larger needle can cause less pain and less tissue injuries when puncturing the skin and arteriovenous access such as arteriovenous fistula (14) or arteriovenous graft. Because the wound healing time by a hemodialysis needle device having multiple thinner needles is faster compared to that by a larger single needle, complications associated with repetitive needle puncturing for hemodialysis can be minimized. In addition, the overall blood flow rate by multiple needles would be compatible or even better than that by a single larger needle.

### CROSS-REFERENCE TO RELATED APPLICATION

USPTO application Ser. No. 11/420,767 and 11/594,321

### FEDERALLY SPONSORED RESEARCH

None.

### SEQUENCE LISTING

None.

### BACKGROUND

1. Field of Invention

This invention relates to a hemodialysis needle device designed to minimize pain and tissue injury when it punctures the skin and arteriovenous accesses such as arteriovenous fistula or arteriovenous graft for hem dialysis.

2. Prior Art

When kidney function fails due to various reasons such as diabetes mellitus or atherosclerosis, the patient can not excrete bodily wastes through the urine. One of the most common treatment procedure to get rid of the wastes of body is hemodialysis. The principle of hemodialysis is to artificially filter the blood of the patient containing the bodily wastes by a hemodialysis machine. Through a large needle, the patient's blood is drained out of the arteriovenous access that is usually created in the arm surgically. After the blood has been filtered by the hemodialysis machine, the blood will be infused back to the patient through another large needle that is placed into the arteriovenous access at a different site from the needle for blood draining.

The needles currently used for puncturing the skin to place the needle into the arteriovenous access for hemodialysis has a size of 14 to 15 gauge. And the patients with kidney failure usually undergo hemodialysis 3 times a week.

Because of the large sized needle that punctures the delicate wall of the arteriovenous access numerous times, the patient not only suffers from the pain but also develops many problems from the tissue injuries and damages of the arteriovenous access that often lead to thrombosis, infection, malfunction and the closure of the arteriovenous access. Needless to say, the cost to fix the malfunctions of the arteriovenous access as well as the pain and suffering of the patients are tremendous.

### BACKGROUND OF INVENTION—OBJECTS AND ADVANTAGES

In my previous invention (USPTO application Ser. No. 11/420,767), I have introduced a new concept of a needle device: Instead of using a single needle, using multiple thinner needles can reduce pain and tissue injuries, and improve the time to heal from the injury by needle puncture. Also in another invention of mine (USPTO application Ser. No. 11/594,321), a biopsy needle device was introduced using the same concept: multiple thinner needles instead of a larger single needle.

This concept is based on the 2 biological facts:

### 1. A larger puncture wound takes longer time to heal than a smaller puncture wound by a thinner and smaller needle, simply because of more tissue injury by the bigger needle puncture.

2. The time period to heal a single puncture wound is the same as the time period to heal multiple simultaneous puncture wounds, as long as the extent of the puncture wound is the same. This is because the healing process of the multiple puncture wounds takes place at the same time at each wound. In other words, it does not matter whether the number of the wound is 1 or 2, the healing occurs at each wound simultaneously, and the healing will complete at the same time.

Therefore, the tissue injuries caused by 2 punctures wounds by smaller and thinner needles is less than the injury by a single needle of larger size.

As a result, the healing time will be shorter in the puncture wound by 2 thinner needles than that by a larger single needle.

This concept is particularly useful in applying for the needles used for hemodialysis. For example, repetitive injuries by a large 14 gauge needle can be minimized by using a needle having two 18 gauge needles. Because of 2 needles are used simultaneously, the overall blood flow rate would be compatible or even better than that of a single 14 gauge needle.

### SUMMARY

A hemodialysis needle device that has multiple thinner needles instead of a single larger needle can cause less pain and less tissue injuries when puncturing the skin and arteriovenous access such as arteriovenous fistula or arteriovenous graft. Because the wound healing time by a hemodialysis needle device having multiple thinner needles is faster compared to that by a larger single needle, complications associated with repetitive needle puncturing for hemodialysis can be minimized. In addition, the overall blood flow rate by multiple needles would be compatible or even better than that by a single larger needle.

### DRAWINGS—FIGURES

FIG. 1 is a perspective view of a hem dialysis needle device having 2 needles mounted on a needle hub.

FIG. 2 is a perspective view of an forearm of a patient undergoing hem dialysis.

FIG. 3 is a perspective view of different embodiment of a hemodialysis needle coupled with a stylet device.

FIG. 4 is a perspective view of the needle of FIG. 3 after removing the stylet device.

FIG. 5 is a perspective view of a stylet device.

FIG. 6 is a perspective view of a hemodialysis needle device without a wing for gripping.

FIG. 7 is a perspective posterior view of the needle device of FIG. 6 having a spiral lock in the posterior opening to secure connection with a connecting catheter.

FIG. 8 is a perspective view of a connecting catheter having a spiral lock in the end of the catheter to secure the connecting with the needle device of FIG. 7.

### DRAWINGS—REFERENCE NUMERALS

9. A needle mounted on a needle hub.
10. A needle hub.
11. A wing for gripping.
12. An artery of the forearm.
13. A vein of the forearm.
14. An arteriovenous fistula
15. A hemodialysis needle inserted into the arteriovenous fistula area to drain the blood out of the patient to the hemodialysis machine.
16. A hemodialysis needle inserted into the venous side of the arteriovenous fistula to return the dialyzed blood back to the patient.
17. A needle with a round tip without cutting edge This needle part also has an extra opening in the side of the needle body to reduce jet pressure of the blood flow and to increase the blood flow rate.
18. A stylet device to be coupled with the needle device.
19. A body part of the stylet device.
20. A spiral lock in the posterior opening of the needle for secure connection.
21. A spiral lock in the end of a connecting catheter to connect with the needle securely.
22. A connecting catheter.
23. A side wall opening of the needle.

### DETAILED DESCRIPTION

FIG. 1 is a hem dialysis needle device having two 18 gauge needles (9) mounted on a needle hub (10). A plastic wing (11) is attached for gripping and controlling the needle device.

A connecting catheter is attached at the end of the needle hub.

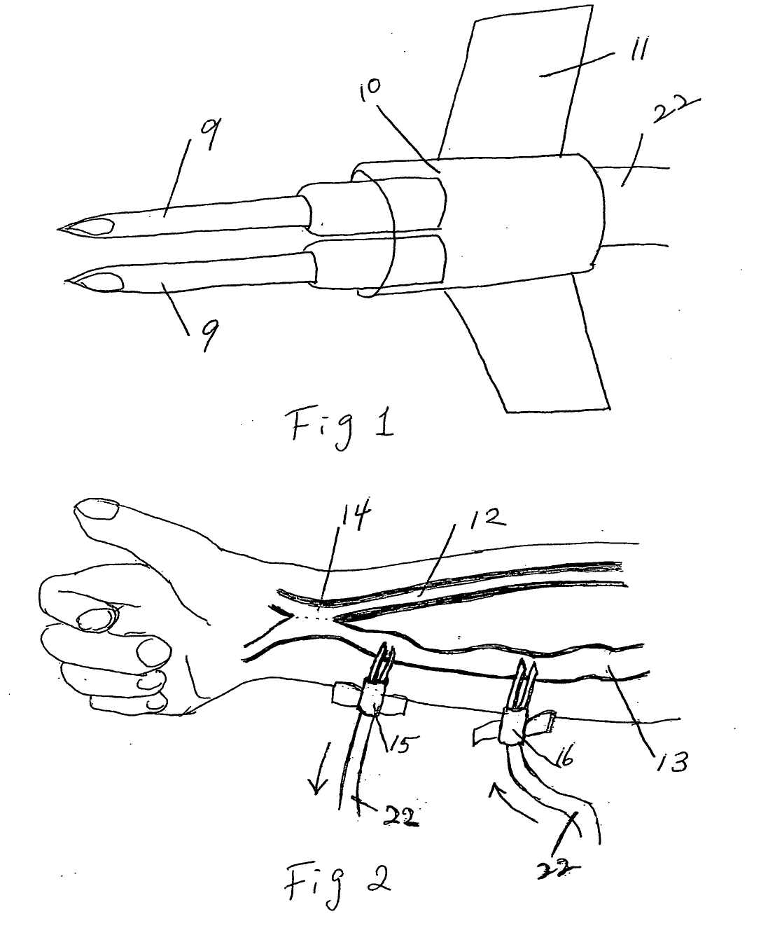

FIG. 2 is a simple drawing of a patient's forearm undergoing hemodialysis. The patient with kidney failure needs to have an arteriovenous access (14) where the hemodialysis needle is inserted for draining the blood (15) at a very high blood flow rate. Another needle is also punctured into the venous side (16) of the arteriovenous access to infuse back the dialyzed (filtered) blood to the patient. In this patient, an arteriovenous fistula (14) is created for arteriovenous access. In order to dialyze the blood in several hours, the blood flow rate through the needles has to be high.

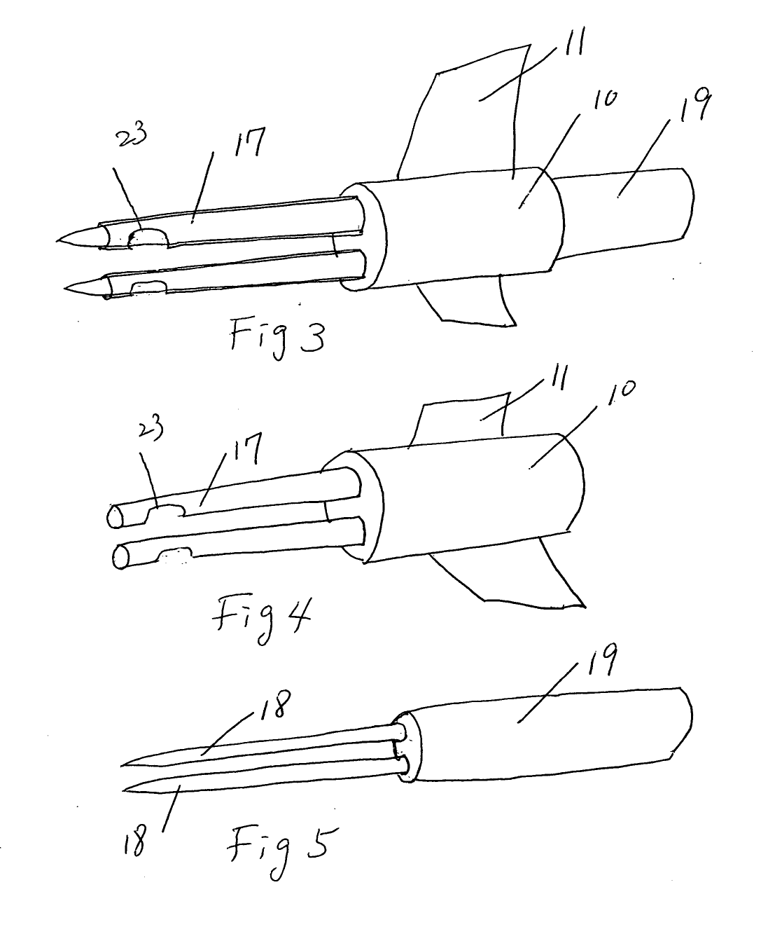

FIG. 3 is a perspective view of a different embodiment of a needle device. In this embodiment, a stylet device is used to couple the needle device to minimize the tissue injury by the cutting edge of the needle tip when puncturing. The tip of this needle is round without a cutting edge. This needle device also has an opening in the side wall of the needle (23) to reduce the jet pressure from the high blood flow rate by reducing resistance of the lumen, and to increase the blood flow rate.

FIG. 4 is the needle device of FIG. 3 after the stylet device has been removed.

FIG. 5 is a stylet device having 2 stylets (18) mounted on the body part (19).

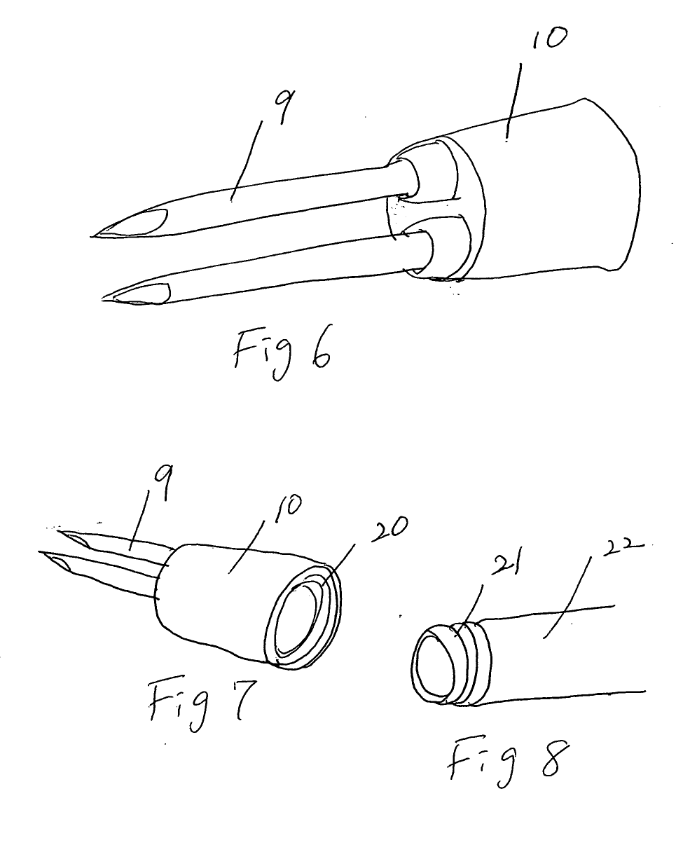

FIG. 6 is a different embodiment of a needle without a grip wing. This needle concept can be applied for other purposes, such as for venous puncture, arterial puncture, or for injecting liquids into a target organ.

FIG. 7 is a perspective posterior view of the needle device having a spiral lock in the posterior opening (20) to securely connect the catheter (22).

FIG. 8 is a perspective view of a catheter (22) having a spiral lock system (21).

### Operation

After identifying the arteriovenous (AV) access (14) in the forearm, a nursing staff make preparation for hem dialysis. The skin area is cleaned and sterilized with alcohol or other antiseptics. The nurse grips the wing (11) of the hemodialysis needle (FIG. 1) that is inserted into the venous side of the arteriovenous access (14). A draining needle is punctured the skin and inserted into the arteriovenous access (15), and another needle is inserted to infuse the blood back (16) after hemodialysis. The blood drained out of the patient through the draining needle and the connecting catheter is sent to the dialysis machine for filtering the bodily wastes. The dialized blood is infused back to the patient by another set of an infusing needle and the connecting catheter.

In a different embodiment of a needle device (FIG. 3) having a stylet coupled with the needle, the stylet (FIG. 5) is pulled back and removed from the needle after the needle device is inserted into the vein. By avoiding a cutting edge of the needle, the tissue injury may be minimized. A side wall opening (23) is created to reduce the blood flow resistance and jet pressure.

In another embodiment, no gripping wing is attached (FIG. 6). A spiral lock (20) is created in the posterior opening of the needle for secure connection with a catheter (22).

I claim:

1. A hemodialysis needle or cannula device having a plurality of a needle mounted on a hub.

2. The needle device of claim 1, wherein said needle device has various kinds of size, length, shape, style, configuration, materials, and color. For example, the needle can be a cannula made of a plastic material.

3. The needle device of claim 2, wherein said needle device's needle part has a various shape or style with or without cutting edge. For example, an extra opening can be created on the side wall of the needle to reduce jet pressure and to increase the blood flow rate.

4. The needle device of claim 3, further including a stylet device coupled with the needle device.

5. The needle device of claim 3, further including an accessories such as a wing attached on the needle device for gripping and controlling the needle device, or a connecting catheter attached to the needle device, or a spiral lock in the posterior opening of the needle hub.

6. The needle device of claim 4, further including an accessories such as a wing attached on the needle device for gripping and controlling the needle device, or a connecting catheter attached to the needle device, or a spiral lock in the posterior opening of a needle hub.

7. The needle device of claim 1, wherein said needle device can be used for other purposes of puncturing to drain or injecting liquids, such as for venous puncturing, arterial puncturing, or puncturing other organs such as lung, liver, peritoneum, pleural membrane, or eyeball.
